(19) United States
(12) Patent Application Publication
(10) Pub. No.: US 2008/0161874 A1
(43) Pub. Date: Jul. 3, 2008

(54) Systems and methods for a trial stage and/or long-term treatment of disorders of the body using neurostimulation

(75) Inventors: Maria E. Bennett, Lyndhurst, OH (US); Julie Grill, Chapel Hill, NC (US); Tina E. Lechman, Chagrin Falls, OH (US); Joseph J. Mrva, Euclid, OH (US); Robert B. Strother, Willoughby Hills, OH (US); Geoffrey B. Thrope, Willoughby Hills, OH (US); Therese M. Zmina, Willoughby, OH (US); Steven M. Galecki, Concord, OH (US); Danny R. Pack, Avon Lake, OH (US)

Correspondence Address: RYAN KROMHOLZ & MANION, S.C., POST OFFICE BOX 26618, MILWAUKEE, WI, 53226, US

(73) Assignee: NDI Medical, Inc.

(21) Appl. No.: 11/981,424

(22) Filed: Oct. 31, 2007

Related U.S. Application Data
(63) Continuation-in-part of application No. 11/729,333, filed on Mar. 28, 2007.
Application No. 11/729,333 is a continuation-in-part of application No. 11/149,654, filed on Jun. 10, 2005.
Application No. 11/149,654 is a continuation-in-part of application No. 11/595,556, filed on Nov. 10, 2006.
Application No. 11/595,556 is a continuation-in-part of application No. 10/777,771, filed on Feb. 12, 2004, now Pat. No. 7,120,499.
(60) Provisional application No. 60/578,742, filed on Jun. 10, 2004.

(51) Int. Cl. A61N 1/00

(52) U.S. Cl. 607/39

(57) ABSTRACT

Systems and methods screen and/or treat disorders of the body using neurostimulation. A trial system implants a temporary or permanent percutaneous lead and couples the lead to an external pulse generator. The external pulse generator supplies a prescribed stimulation regime through the lead to a targeted tissue region. If an improvement in the treated disorder is achieved, use of the trial system may be continued, or an implantable system may be implanted.

### RELATED APPLICATIONS

This application is a continuation-in-part of co-pending U.S. patent application Ser. No. 11/729,333, filed Mar. 28, 2007, and entitled “Systems and Methods for Bilateral Stimulation of Left and Right Branches of the Dorsal Genital Nerves to Treat Urologic Dysfunctions, which is a continuation-in-part of co-pending U.S. patent application Ser. No. 11/149,654, filed Jun. 10, 2005, and entitled “Systems and Methods for Bilateral Stimulation of Left and Right Branches of the Dorsal Genital Nerves to Treat Dysfunctions Such as Urinary Incontinence,” which claims the benefit of U.S. Provisional Patent Application Ser. No. 60/578,742, filed Jun. 10, 2004, and entitled “Systems and Methods for Bilateral Stimulation of Left and Right Branches of the Dorsal Genital Nerves to Treat Dysfunctions, Such as Urinary Incontinence.”

This application is also a continuation-in-part of co-pending U.S. patent application Ser. No. 11/595,556, filed Nov. 10, 2006, and entitled “Portable Assemblies, Systems, and Methods for Providing Functional or Therapeutic Neurostimulation,” which is a continuation-in-part of U.S. patent application Ser. No. 10/777,771, filed Feb. 12, 2004, (now U.S. Pat. No. 7,120,499), and entitled “Portable Percutaneous Assemblies, Systems, and Methods for Providing Highly Selective Functional or Therapeutic Neurostimulation.” Each of the preceding applications is incorporated herein by reference.

### STATEMENT REGARDING FEDERALLY SPONSORED RESEARCH

This invention was made with government support under one or more grant numbers: 1R43AG021851-01 awarded by the National Institutes of Health, through the National Institute of Aging; 1R43AG022292-01 awarded by the National Institutes of Health, through the National Institute of Aging; and 1R43AR052211-01 awarded by the National Institutes of Health, through the National Institute of Arthritis and Musculoskeletal and Skin Diseases. The Government has certain rights in the invention.

### FIELD OF THE INVENTION

This invention relates to systems and methods for stimulating tissue in animals, including humans, and more specifically to systems and methods used for a trial stage and/or treatment of disorders using neurostimulation.

### BACKGROUND OF THE INVENTION

Many millions of people throughout the world suffer from a variety of pelvic region or pelvic floor disorders (or dysfunctions). Pelvic region disorders are generally understood to include indications under a variety of names, such as urinary incontinence (including at least stress and urge incontinence), overactive bladder, neurogenic bladder, micturition disorders (including at least urinary retention), defecation disorders (including at least fecal incontinence and constipation), sexual disorders (including at least erection, ejaculation, orgasm, vaginal lubrication, arousal (pleasure), and engorgement disorders), pelvic floor muscle disorders, prostate disorders, and pelvic pain disorders (including at least interstitial cystitis and painful bladder syndrome).

As one example, thirteen million Americans suffer from various types of urinary incontinence. The most prevalent type of urinary incontinence is called stress incontinence. Stress incontinence is, characterized by the unintended emission of urine during everyday activities and events, such as laughing, coughing, sneezing, exercising, or lifting. These activities and events cause an increase in bladder pressure resulting in loss of urine due to inadequate contraction of the sphincter muscle around the outlet of the bladder.

Another prevalent type of urinary incontinence is called urinary urge incontinence. Urge incontinence is characterized by a strong desire to urinate, followed by involuntary contractions of the bladder, which is often identified as a symptom of an overactive bladder. Because the bladder (i.e., the detrusor muscle) actually contracts, urine is released quickly, making it impossible for urge incontinence sufferers to predict when the problem will occur. Urge incontinence can be caused by infections, sphincter disorders, or nervous system disorders that affect the bladder. Many people encounter a combination of bladder control disorders.

The prostate is a firm chestnut sized gland in males that lies immediately below the bladder and surrounds the urethra. Prostate disorders can lead to prostate swelling, pain, urinary incontinence, urinary retention, and sexual dysfunction.

In the absence of consistent causes or origins for chronic prostate disorders, improvement in quality of life and a reduction in symptoms are the usual goals of therapy. The most common treatment of prostate disorders includes pharmacologic treatments (antibiotics, anti-inflammatory agents, alpha blockers, anti-spasmodics, analgesics, allopurinol, and muscle relaxants).

Sexual dysfunction includes an assortment of problems, including erectile dysfunction, orgasmic dysfunction, premature ejaculation, and lack of lubrication, and can affect both women and men. A wide range of options exist for the restoration of sexual function. Treatments include everything from medications, simple mechanical devices, psychological counseling, external stimulators, and surgically implanted neurostimulation devices.

The number of people suffering from the variety of pelvic region disorders is on the rise as the population ages. Various treatment modalities for these pelvic region disorders have been developed. These modalities typically involve drugs, surgery, or both. Some are intended to treat the disorder, while others are only intended to deal with its consequences.

One present surgical modality for the treatment of incontinence involves the posterior implantation of electrodes percutaneously through the S3 spinal foramen into the muscles and ligaments near the right or left sacral nerves (INTERSTIM® Treatment, Medtronic). The electrodes are connected to a remote neurostimulator pulse generator implanted in a subcutaneous pocket on the right hip to provide unilateral spinal nerve stimulation. This surgical procedure near the spine is complex and requires the skills of specialized medical personnel. Furthermore, in terms of outcomes, the modality has demonstrated limited effectiveness and limited reliability.

A recently proposed alternative surgical modality entails the implantation of an integrated neurostimulator and bi-polar electrode assembly (called the BION® System from Advanced Bionics Corporation). The BION is implanted perineally, and is passed through a 12 gauge hypodermic needle and into tissue near the pudendal nerve on the left side adjacent the ischial spine. See, e.g., Mann et al, U.S. Pat. No. 6,941,171. The clinical effectiveness of this modality is not known.

Some surgical procedures are not designed to include a test or trial stage to determine the efficacy of electrical stimulation of a targeted nerve or pelvic region. A stimulating needle may be incorporated into the surgical implantation process to determine the desired location for a stimulating electrode, but the stimulating needle does not allow for the physician and patient to “test” the electrical stimulation for an extended trial period, such as hours, days, weeks, or months, prior to the more involved implantation of the neurostimulation system.

Other surgical procedures include a test phase that is just as invasive as the implantation of a long-term, fully implanted neurostimulation system. The invasiveness of the test phase may be a deterrent to acceptance of the neurostimulation treatment by those who may benefit from it the most.

There remains a need for less complicated systems and methods that can screen and/or treat pelvic region disorders, the systems and methods including a trial (i.e., screening) stage or phase to determine the efficacy of electrical stimulation to effectively treat the disorders. Based upon the outcome of a simple trial stage, an informed decision can be made if a fully implantable neurostimulation system is warranted.

### SUMMARY OF THE INVENTION

The invention provides improved systems and methods used for a trial stage (screening) and/or treatment of disorders of the body using neurostimulation.

One aspect of the invention provides systems and methods for screening and/or treating pelvic region disorders, including, but not limited to urinary incontinence, overactive bladder, neurogenic bladder, micturition disorders, defecation disorders, sexual disorders, prostate disorders, pelvic floor muscle disorders, and pelvic pain.

The methods and associated components may include providing an external pulse generator, providing a percutaneous lead including a connector at or near a proximal portion, and an electrode at or near a distal portion, the electrode being electrically coupled to the connector, inserting the distal portion of the percutaneous lead into tissue near-midline over the pubic symphysis, positioning the electrode at a target site between the pubic symphysis and the clitoris of a female or the base of the penis of a male, coupling the percutaneous lead to the external pulse generator, and operating the external pulse generator to convey stimulation waveforms to the electrode to screen and/or treat the pelvic region disorder.

The target site between the pubic symphysis and the clitoris of a female or the base of the penis of a male may comprise an adipose tissue region innervated with one or more nerves. The stimulation waveforms conveyed to the electrode are able to affect stimulation of the left or right branches of the dorsal genital nerves. Alternatively, the stimulation waveforms conveyed to the electrode affect bilateral stimulation of the left and right branches of the dorsal genital nerves.

The step of providing an external pulse generator may include providing a removable and replaceable carrier adapted to be worn by a user. The step may also include providing a removable and replaceable electronics pod to electrically couple to the carrier, the electronics pod comprising circuitry adapted to generate electrical stimulation current patterns to be delivered through the percutaneous lead and to the electrode.

The electronics pod also comprises a power input bay adapted to receive a self-contained, limited life, disposable, smart power source that can be released and replaced for a prescription period, the power source including-circuitry to provide power source information to the electronics pod and/or receive power source information from the electronics pod.

In one aspect of the invention, a test stimulator is provided, the test stimulator comprising a hand-held, single use, sterile, and disposable device including test stimulation generating circuitry and a non-rechargeable and non-replaceable battery adapted to keep the test stimulator operational for a predetermined time.

The test stimulator may be coupled to the proximal portion of the percutaneous lead and operated to deliver test stimulation to the electrode to confirm the electrode is positioned at the target site. The electrode may be adjusted if necessary to confirm the electrode is positioned at the target site. After use, the test stimulator may be disconnected from the lead and discarded.

In an additional aspect of the invention, a method for providing neurostimulation to tissue comprises the steps of providing a percutaneous lead including a proximal portion and a distal portion, the distal portion including one or more electrodes, providing an external pulse generator adapted to electrically couple to the percutaneous lead and one or more electrodes, the external pulse generator comprising a carrier adapted to be worn by a user, and providing a removable and replaceable electronics pod to electrically couple to the carrier, the electronics pod comprising circuitry adapted to generate electrical stimulation current patterns to be delivered through the percutaneous lead and to the at least one electrode to stimulate tissue.

The electronics pod may also comprise a power input bay adapted to receive a self-contained, limited life, disposable, smart power source that can be released and replaced for a prescription period, the power source including circuitry to provide power source information to the electronics pod and/or receive power source information from the electronics pod.

The method may further include providing instructions for use prescribing the release and replacement of the power source according to a prescribed power source replacement regime, the prescribed power source replacement regime comprising the replacement of the power source on a prescribed repeated basis similar to administering a pill under a prescribed pill-based medication regime, and providing a supply of power sources, each power source comprising a dose of power for the circuitry for administration according to the prescribed power source replacement regime.

The method may further include implanting the distal portion of the percutaneous lead on, in, or near a targeted tissue region, which may be performed without fluoroscopy, coupling the percutaneous lead to the external pulse generator, inserting a power source into the power input bay, the power source circuitry providing power source information to the electronics pod and/or receiving power source information from the electronics pod, and operating the external pulse generator to convey stimulation waveforms to the targeted tissue region to provide neurostimulation.

In one aspect of the invention, the targeted tissue region comprises the left and/or right branches of the dorsal genital nerves, the pudendal nerve and/or its branches, the perineal nerves, and/or its branches, the urethral nerves, and/or its branches, and/or the sacral nerves.

In another aspect of the invention, the power source information comprises the power capacity of the power source, or the total power usage provided to date by the power source, or the total power capacity remaining in the power source, or a unique identification to identify the power source, or any combination.

The supply of power sources may comprise a power source organizer that includes a compartment for each prescribed repeated basis, the compartment adapted to hold one or more power sources.

In yet another aspect of the invention, systems and methods provide a neurostimulation system for screening and/or treatment of pelvic region disorders. The systems and methods comprise a percutaneous lead including a proximal portion and a distal portion, the proximal portion including a connector, the distal portion able to reside in adipose tissue and including an electrode, the electrode being electrically coupled to the connector.

An external pulse generator is provided and is adapted to electrically couple to the percutaneous lead, the external pulse generator comprising a removable and replaceable carrier adapted to be worn by a user, the carrier including a tissue facing surface having pressure sensitive adhesive to removably secure the carrier to the user's skin.

A removable and replaceable electronics pod electrically couples to the carrier, the electronics pod containing circuitry and adapted to generate electrical stimulation current patterns to be delivered through the percutaneous lead and to the electrode to stimulate tissue, and a power input bay is adapted to receive a self-contained, limited life, disposable, smart power source that can be released and replaced for a prescription period, the power source including circuitry to provide power source information to the electronics pod and/or receive power source information from the electronics pod.

In yet another aspect of the invention, systems and methods provide a kit of devices to screen and/or treat pelvic region disorders. The kits may include a variety of components and instructions for use. A kit may include a percutaneous lead including a proximal portion, and an electrode at or near a distal portion.

A kit may also include an external pulse generator, the external pulse generator comprising a carrier adapted to be worn by a user and a removable and replaceable electronics pod adapted to be coupled to the carrier, the electronics pod containing circuitry and adapted to generate electrical stimulation current patterns to be delivered through the percutaneous lead and to the electrode to stimulate tissue, the electronics pod including a power input bay adapted to receive a self-contained, limited life, disposable, smart power source that can be released and replaced for a prescription period, the power source including circuitry to provide power source information to the electronics pod and/or receive power source information from the electronics pod.

A kit may also include one or more power sources, each power source comprising a dose of power for the circuitry for administration according to a prescribed power source replacement regime. Instructions may be provided for use prescribing the release and replacement of the power source according to the prescribed power source replacement regime, the prescribed power source replacement regime, the regime comprising the replacement of the power source on a prescribed repeated basis similar to administering a pill under a prescribed pill-based medication regime.

A kit may also include instructions for implanting the electrode in tissue in a region at or near a pubic symphysis, coupling the lead to the external pulse generator, and stimulating the left and/or right branches of the dorsal genital nerves by conveying electrical stimulation waveforms from the external pulse generator to the electrode to screen and/or treat the pelvic region disorders.

In yet a further aspect of the invention, systems and methods comprising a neurostimulation system is provided. The systems and methods may comprise a percutaneous lead including a proximal portion, and an electrode at or near a distal portion, a carrier adapted to be worn by a user, a removable and replaceable electronics pod, the electronics pod containing circuitry and adapted to generate electrical stimulation current patterns to be delivered through the percutaneous lead and to the electrode to stimulate tissue, an electrical connection region on the electronics pod to electrically couple to a mating electrical connection region on the carrier, and a power input bay adapted to receive a self-contained, limited life, disposable, smart power source that can be released and replaced for a prescription period, the power source including circuitry to provide power source information to the electronics pod and/or receive power source information from the electronics pod.

Instructions for use may be provided, the instructions prescribing the release and replacement of the power source according to a prescribed power source replacement regime, the prescribed power source replacement regime comprising the replacement of the power source on a prescribed repeated basis similar to administering a pill under a prescribed pill-based medication regime, and one or more power sources may be provided, each power source comprising a dose of power for the circuitry for administration according to the prescribed power source replacement regime.

The power source information may comprise the power capacity of the power source, or the total power usage provided to date by the power source, or the total power capacity remaining in the power source, or a unique identification to identify the power source, or any combination.

A supply of power sources may be provided, the supply of power sources comprising a power source organizer that includes a compartment for each prescribed repeated basis, the compartment adapted to hold one or more power sources.

Other features and advantages of the inventions are set forth in the following specification and attached drawings.

### BRIEF DESCRIPTION OF THE DRAWINGS

FIG. 1 is a perspective view of a trial system for testing the efficacy of electrical stimulation, and/or for longer term treatment of a wide variety of disorders in animals, including humans.

FIG. 2A is an exploded side view of the trial system shown in FIG. 1 in use, showing the external pulse generator coupled to the percutaneous lead, the lead extending below the skin surface to a targeted tissue region or regions.

FIG. 2B is a plane view of the trial system shown in FIGS. 1 and 2A in use, showing the external pulse generator removably adhered to the user's skin, and the percutaneous lead extending from the external pulse generator to the point at which the lead passes through the skin and ends with one or more electrodes to stimulate a targeted tissue region.

FIG. 3 is a perspective view with a partial cutaway showing the power source housing and internal and external components.

FIG. 4A is a perspective view of the trial system comprising an external pulse generator of the type shown in FIG. 1 in association with a prescribed supply of replacement power sources and instructions for using the a system, including the powering of the neurostimulation therapy by inserting a fresh power source, just as an individual on a medication regime “doses” their medication therapy by taking a pill.

FIG. 4B is a perspective view of a power source pill case or organizer to aid in patient compliance of the prescribed neurostimulation regime.

FIGS. 5A through 5C are perspective views of examples of styles of leads and electrodes that may be used with the system shown in FIGS. 1 and 2.

FIG. 6 is a plan view of a test stimulator system used with the present invention, including the hand-held test stimulator, a return patch electrode, and an extension cable.

FIG. 7A is a plane view of an implantable neurostimulation system including an implanted pulse generator and an implanted lead, and a patient controller-charger to communicate wirelessly with the implanted pulse generator.

FIG. 7B is an anterior anatomical view of the implantable neurostimulation system, and showing one example of a possible placement for the implantable pulse generator and lead.

FIGS. 8A and 8B are anterior anatomic views of the system shown in FIGS. 7A and 7B after implantation of the pulse generator in a pelvic region and the lead/electrode in an adipose tissue region at or near the pubic symphysis.

FIG. 9 is an anterior anatomic view of the pelvic girdle in a human.

FIG. 10 is a lateral section view of the pelvic girdle region shown in FIG. 9.

FIG. 11A is an inferior view of the pelvic girdle region shown in FIG. 9.

FIG. 11B is an anatomical view showing the distal portions of the dorsal genital nerve branches near the pubic symphysis and the body of the clitoris.

FIGS. 12 through 18 illustrate steps incorporating the trial system, including implanting a lead in a targeted tissue region and coupling the lead to the external pulse generator.

FIGS. 19 through 22 illustrate steps incorporating the implantable system, including tunneling a lead from the targeted tissue region, coupling the lead to the implantable pulse generator, inserting the pulse generator in a subcutaneous pocket, and testing the implantable system with a clinical programmer.

FIG. 23 is a plane view of an exemplary kit of devices of the present invention to screen and/or treat pelvic region disorders.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DESCRIPTION OF THE PREFERRED EMBODIMENTS

The various aspects of the invention will be described in connection with the treatment of pelvic region disorders. In one exemplary embodiment, urinary disorders are treated by the bilateral stimulation of the left and/or right branches of the dorsal genital nerves using a single lead implanted in adipose or other tissue in the region at or near the pubic symphysis. That is because the features and advantages of the invention are well suited for this purpose. Still, it should be appreciated that the various aspects of the invention can be applied in trial and/or treatment of any of the pelvic region disorders described, and also in other locations in the body, and to a wide variety of nerves and anatomical locations, to achieve other objectives as well.

### I. The Trial System

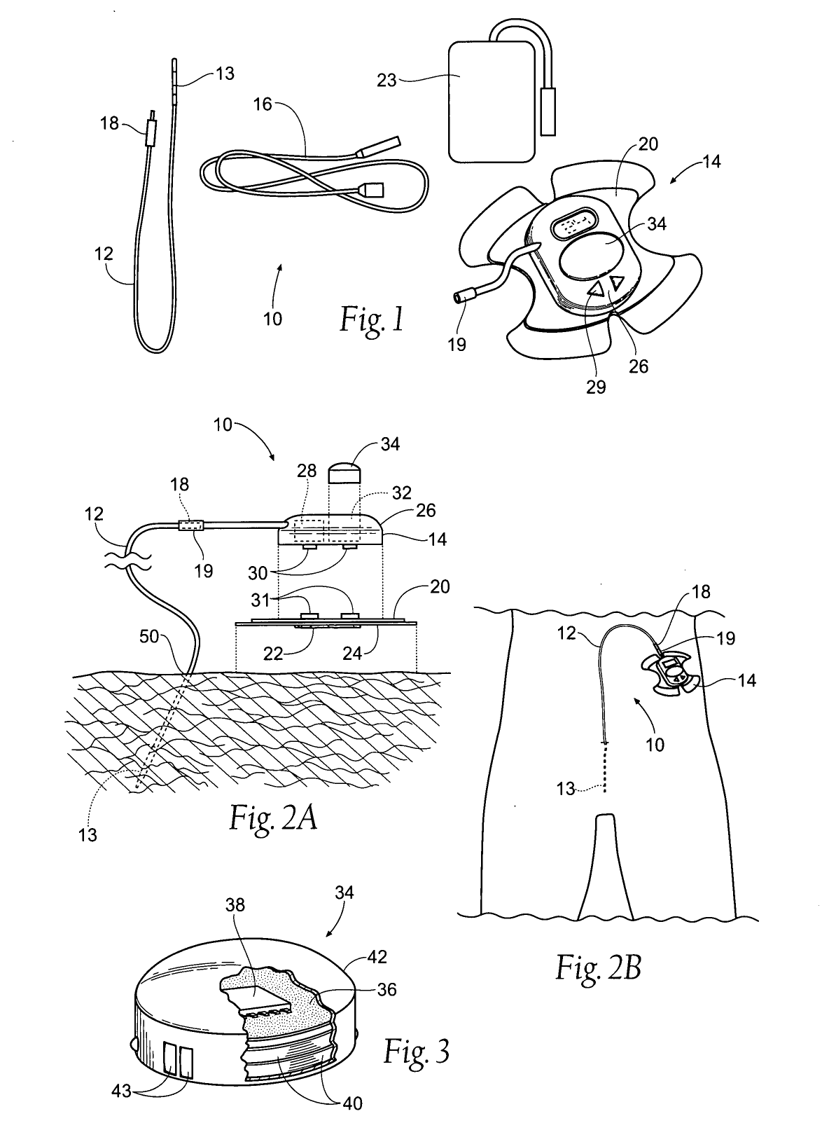

FIG. 1 shows a trial system 10 for determining the efficacy of electrical stimulation to effectively treat one or more disorders. The trial system provides electrical stimulation using a temporary or permanent percutaneous lead 12 and a temporary external pulse generator 14 to evaluate if the electrical stimulation is effective at treating the disorder, and if the patient is a suitable candidate for extended placement of an implantable neurostimulation system 60 (to be described in greater detail later). It is to be appreciated that the term “stimulation” includes excitation, modulation, and inhibition (blocking) of action potentials in nerves.

A lead extension 16 may also be included with the trial system 10 to extend the length of the lead 12 to allow for a greater possible range of locations for the external pulse generator 14 to be located. The trial stage can be conducted, e.g., during an hour, day, week, or month period (or shorter or longer, as desired). If the patient is a suitable candidate, the second phase can be scheduled, which comprises the implantation of a neurostimulation system.

The trial system 10 may also comprise a standalone system, i.e., the trial system 10 may take the place of an implantable system if implantation of an implantable system is not desirable for any number of reasons.

An external pulse generator 14 can be used of the type described in U.S. Pat. No. 7,120,499, issued Oct. 10, 2006, and entitled “Portable Percutaneous Assemblies, Systems, and Methods for Providing Highly Selective Functional or Therapeutic Neurostimulation,” which is incorporated herein by reference. Optionally, an external pulse generator 14 can be used of the type described in co-pending U.S. patent application Ser. No. 11/595,556, filed Nov. 10, 2006, and entitled “Portable Assemblies, Systems, and Methods for Providing Functional or Therapeutic Neurostimulation,” which is also incorporated herein by reference.

As shown in FIGS. 1 through 2B, the external pulse generator 14 comprises a patch or carrier 20. The carrier 20 can be readily worn on the skin, e.g., by use of a pressure-sensitive adhesive, without discomfort and without affecting body image on, for example, an arm, a leg, or torso of an individual. The external pulse generator 14 can then be removed after the trial period. In place of worn on the skin, the patch or carrier 20 may also be readily worn, e.g., in a shirt pocket, or carried by the patient on a belt, or secured to clothing, a bed, or to movable devices to allow for patient mobility.

As shown in FIGS. 2A and 2B, the percutaneous lead 12 is passed through the skin to position the one or more electrodes 13 at or near the desired location. The proximal portion of the lead 12, which may be outside the body and includes the plug 18, is coupled to the connector 19 of the external pulse generator 14. The lead 12 does not need to be subcutaneously tunneled to a remote site, although depending on the implantation site, it may be desirable to tunnel all or at least a portion of the lead 12.

In one embodiment, the carrier 20 may include an integral return electrode 22 on its tissue facing surface 24. In an alternative embodiment, the patch return electrode 23 is included, or is otherwise available, to be coupled to the external pulse generator 14, to provide a return path for the stimulation waveforms.

The carrier 20 carries a removable and replaceable electronics pod 26, which generates the desired electrical stimulation current patterns. The pod 26 houses microprocessor-based, programmable circuitry 28 that generates the stimulus currents, times or sequences the stimulation pulses, monitors system status, and logs and monitors usage, e.g., time and stimulus pulse parameters, to be reported back to a physician. The electronics pod 26 may include user and/or clinician controls 29, or the electronics pod may be configured to couple to a programming/control device, or if desired, to accept wireless RF based commands for both wireless programming and wireless patient control, or any combination control options.

An electrical connection region or contact(s) 30 on the pod 26 electrically couples to a mating connection region or contact(s) 31 on the carrier 20, to couple the circuitry 28 on the pod 26 to the return electrode 22 positioned on the tissue facing surface 24 of or integral with the carrier 20. The electronics pod 26 further includes a power input bay 32, to receive a small, lightweight, self-contained, limited life, disposable smart power source 34, which can be released and replaced as prescribed (see FIG. 3). The power source 34 may provide power and data to the electronics pod 26.

The disposable power source 34 may include circuitry 36 to electronically store information about the power source 34. The circuitry 36 may include a non-volatile memory 38 to store the power source information. The capacity of the power source 34 may be stored, e.g., the power source may identify itself as a one hour power source, or a six hour power source, or a twenty-four hour power source, or a variety of other preconfigured capacities. The circuitry 36 may also identify each unit (e.g., to provide a unique identification, such as serial number), and/or electronically identify the total power usage (service time) provided to date by the power source 34.

The replacement of the power source 34 is the method by which the patient initiates another session of use or episode of treatment. Sessions or episodes of usage/treatment may be interrupted by removing the power source 34, and re-inserting the same power source will resume stimulation; but the total duration of stimulation from that one power source 34 is still limited to the value defined for that power source, e.g., one hour of use, or eight hours, or twelve hours, or twenty-four hours.

The electrical components 36 and battery(s) 40 will be inaccessible to battery replacement. The battery or batteries 40 are secured within a housing 42, such as a non-metallic or only partially metallic molded plastic housing, to aid in handling of the power source 34. The housing 42 may also prevent the use of a power source not intended for the external pulse generator 14. The housing may have a unique shape and/or may include retention features not found on commercially available batteries. The housing may include electrical contact(s) 43 to couple the power source 34 to the pod 26. The housing 42 may also include multiple pieces and may be made inaccessible by sonic welding, gluing, or other permanent fastening methods, to secure the housing together. Even if the battery 40 was replaced, the circuitry 36 of the power source 34 would prevent its reuse.

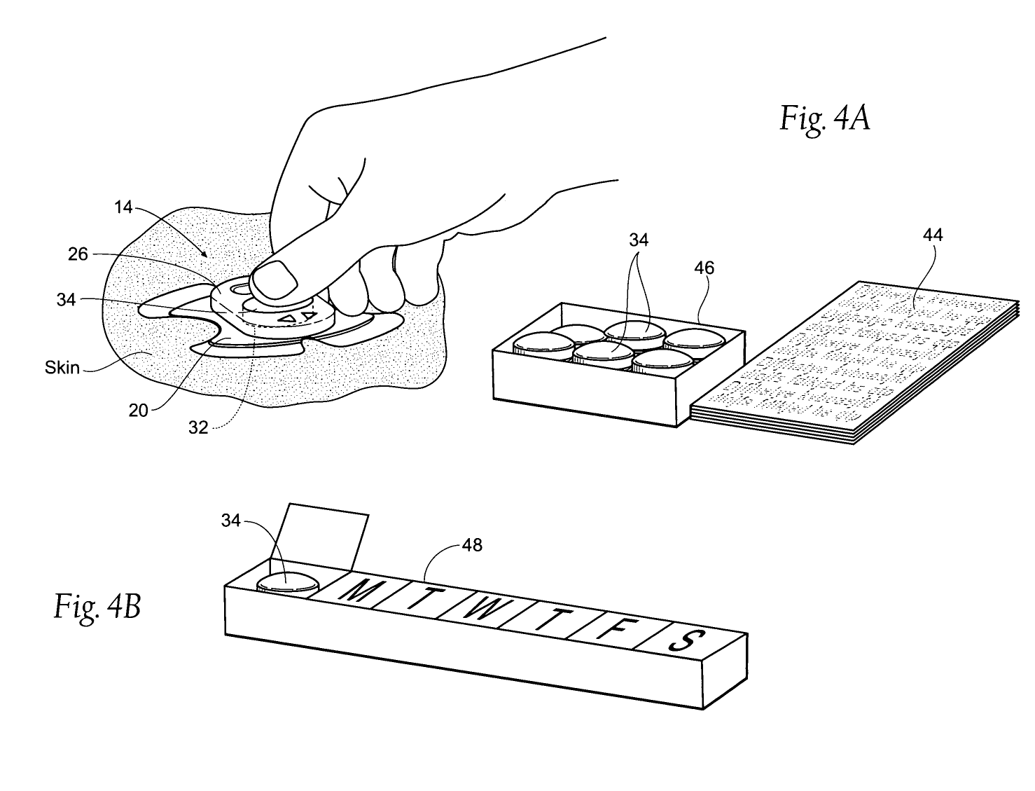

A seen in FIG. 4A, instructions for use 44, along with a supply of disposable power sources 34 for administration according to the prescribed power source replacement regime, i.e., a usage or therapy regime, is intended to be provided, each power source thereby providing a “dose” of power for the circuitry to provide the delivery of the neurostimulation. With the prescribed power source replacement regime (as with a prescribed pill-based medication regime), a caregiver or clinician or physician instructs the patient to remove and replace the disposable power source 34 on a repeated or periodic basis (like taking a dose of medication in pill form) to administer to the circuitry a dose of power so the circuitry can generate a dose of neurostimulation. In this way, the prescribed power sources replacement regime has the effect or flavor of administering a “pill” under a prescribed pill-based medication regime, and not an end-of-life battery timeout.

Typically, as shown in FIG. 4A, a container 46 holding a prescribed number of replacement power sources 34, e.g., seven or fourteen, or more or less, will be provided with the external pulse generator 14, forming a component of the trial system 10. As previously described, the power source 34 can be likened to a “pill,” the pill being a “dose” of power for the stimulation circuitry as a medicine pill provides a dose of medication for a prescribed pill-based medication regime. This gives the patient the responsibility of ownership in treatment, which boosts compliance during the trial period and allows delivery of scheduled stimulation; e.g., every hour, day, week, or month. The container 46 may also be in the form of a seven day (or more or less) pill case or similar organizer 48 that includes one or more compartments to hold one or more disposable power sources, or “pills,” 34, for each day or prescription period to aid in compliance (see FIG. 4B).

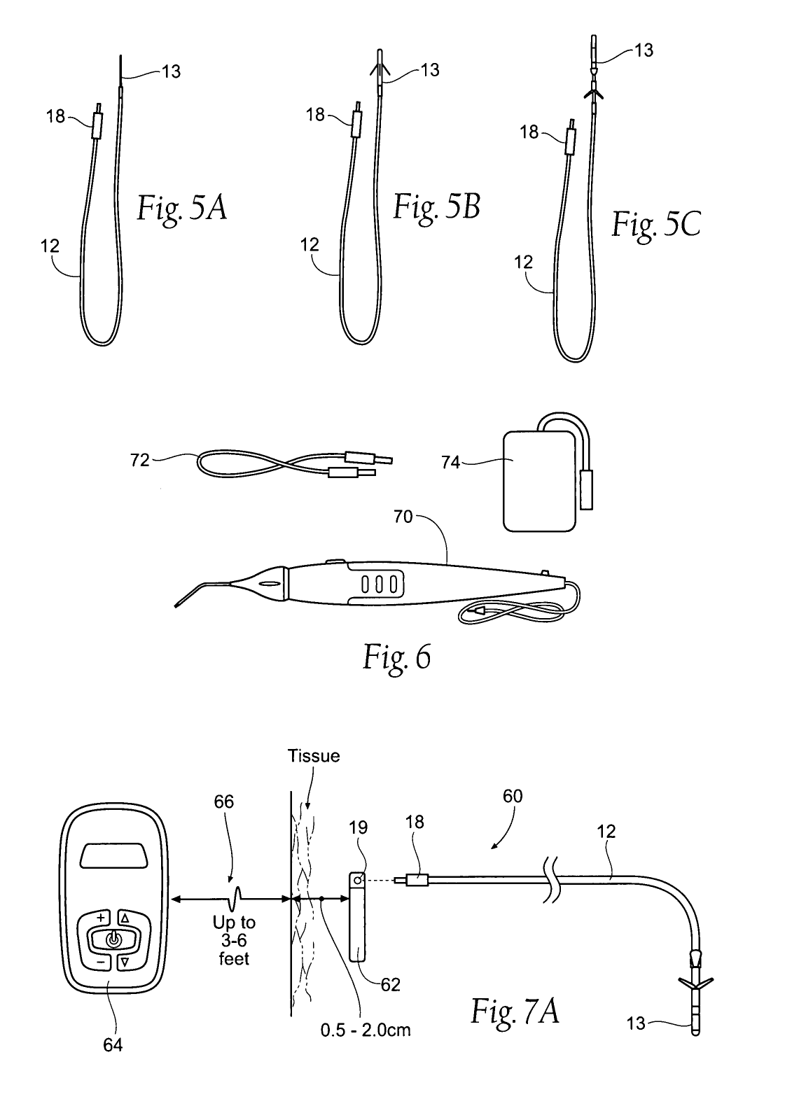

FIGS. 5A through 5C show a non-limiting example of a variety of lead configurations that may be used with the present invention. The percutaneous lead 12 comprises a proximal and a distal end. The proximal end carries a plug 18, which is desirably of an industry-standard size, for coupling to an industry-sized connector 19 on the external pulse generator 14. The distal end includes at least one electrically conductive surface, which will also in shorthand be called an electrode 13. The lead electrically connects the electrode 13 (one or more) to the connector 19, and thus to the external pulse generator 14 itself, while electrically insulating the lead wire from the body tissue except at the electrode(s) 13.

The lead 12 and electrode 13 are sized and configured to be implanted percutaneously in tissue, and to be tolerated by an individual during extended use without pain or discomfort. The comfort is both in terms of the individual's sensory perception of the electrical waveforms that the electrode applies, as well as the individual's sensory perception of the physical or mechanical presence of the electrode and lead. In the case of the mechanical presence, the lead 12 and electrode 13 are desirably “imperceptible.”

The lead 12 may be the same as those included with the second stage implantable system 60 (to be described in greater detail later), or the lead may comprise a more temporary lead to facilitate ease of positioning and/or removal after the trial stage. FIG. 5A shows a fine wire style lead 12 and electrode 13. FIG. 5B shows an intra-muscular or general purpose style lead 12 and electrode 13. FIG. 5C shows a lead 12 and electrode 13 intended for implantation in adipose or other tissue regions. It is to be appreciated that the trial system 10 and the implantable system 60 are not limited to any particular style of lead 12 and/or electrode 13.

A test stimulator 70 may also be included of the type described in co-pending U.S. patent application Ser. No. 11/651,165, filed Jan. 9, 2007, and entitled “Systems and Methods for Intra-Operative Stimulation,” which is incorporated herein by reference. The test stimulator 70 (see FIG. 6) operates to generate stimulation waveforms of the same type as the external pulse generator 14 and an implantable pulse generator 62, as will be described in greater detail later. The test stimulator 70 may be a hand-held, single use, sterile, and disposable device including a non-rechargeable and non-replaceable battery sized to keep the test stimulator operational for a predetermined time, e.g., at least about an hour, and more preferably, at least about six hours. The test stimulator 70 may include a cable 72 to couple the test stimulator 70 to the lead 12. A sterile return electrode, such as a needle or patch electrode 74 may also be also included, which is to be placed in or on the skin of the individual and coupled to the test stimulator 70, to serve as a return path for the stimulation waveforms.

After the lead 12 is implanted and coupled to the external pulse generator, the individual patient wears the external pulse generator 14 for the prescribed trial period. The external pulse generator 14 supplies the prescribed stimulation regime. If an improvement in the treated disorder is achieved, the second phase may be warranted. As will be described in greater detail later, in the second phase, the percutaneous lead 12 may be removed and discarded, or it may be used in the second phase. The lead 12 is tunneled to a subcutaneous pocket 94 sized to hold the implantable pulse generator 62. The implantable pulse generator 62 is connected to the lead 12 and installed in the pocket 94 remote from the electrode 13.

### II. The Implantable System

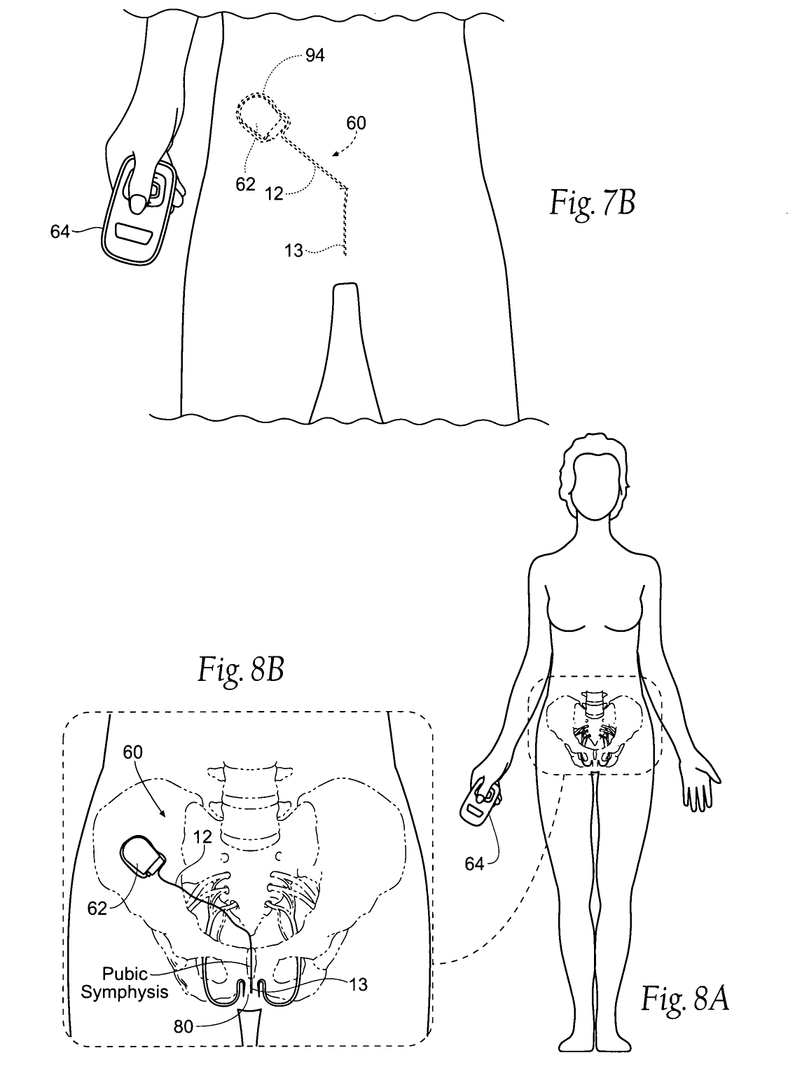

FIGS. 7A and 7B show one embodiment of an implantable neurostimulation system 60 for treating pelvic region disorders in animals, including humans.

The implant system 60 includes an implantable lead 12 having a proximal and a distal end. The proximal end carries a plug 18, which is desirably of an industry-standard size, for coupling to an industry-sized connector 19 on the implantable pulse generator 62. The distal end includes at least one electrode 13 (two are shown). The lead 12 electrically connects the electrode 13 to the connector 19, and thus to the pulse generator 62 itself, while electrically insulating the lead wire from the body tissue except at the electrode(s) 13.

In one embodiment, the lead 12 and electrode 13 are sized and configured to reside with stability in soft or adipose tissue 80 in the lower anterior pelvic region of the body (see FIGS. 8A and 8B). It has been discovered that, when properly placed in this region, a single lead 12 with one or more electrodes 13 is uniquely able to deliver electrical stimulation current simultaneously to one or both of the left and right branches of the dorsal genital nerves, present near the clitoris in a female and near the base of the penis of a male. It is to be appreciated that the term “stimulation” includes excitation, modulation, and inhibition (blocking) of action potentials in nerves.

The implantable system 60 includes the implantable pulse generator 62 of the type described in co-pending U.S. patent application Ser. No. 11/517,056, filed Sep. 7, 2006, and entitled “Implantable Pulse Generator Systems and Methods for Providing Functional and/or Therapeutic Stimulation of Muscles and/or Nerves and/or Central Nervous System Tissue,” which is incorporated herein by reference. The pulse generator 62 includes a circuit that generates electrical stimulation waveforms. An on-board battery (primary or rechargeable) provides the power. The pulse generator 62 also includes an on-board, programmable microprocessor, which carries embedded code. The code expresses pre-programmed rules or algorithms under which the desired electrical stimulation waveforms are generated by the circuit. The small metal case (e.g., titanium) of the pulse generator may also serve as the return electrode for the stimulus current introduced by the lead/electrode when operated in a monopolar configuration.

The pulse generator 62 may define a generally pear-shaped case, although other shapes are possible. The generally pear-shaped case can be described as including a bottom portion defining a curved surface having a radius, inwardly tapering sides, and a top portion being generally flat, as shown in the Figures. This geometry provides a case including a larger end (bottom portion) and a smaller end (top portion) and allows the smaller end of the case to be placed into the skin pocket first, with the larger end being pushed in last.

Both the external pulse generator 14 and the implantable pulse generator 62 can deliver a range of stimulation parameters to the lead 12 and electrode 13, e.g., output current ranges of about 0.5 mA to about 20 mA, pulse duration ranges of about 0.1 microseconds to about 500 microseconds, frequency ranges of about one pulse per second to about 130 pulses per second, and duty cycle ranges from about zero to about 100 percent. The delivered stimulus is an asymmetric biphasic waveform with zero net DC (direct current).

Testing has suggested that OFF times longer than 10 seconds or ON times shorter than 10 seconds may lead to a decrease in the effectiveness of stimulation. Further, increasing ON times to 20 to 40 seconds may increase the effectiveness of stimulation.

The pulse generator 62 is sized and configured to be implanted subcutaneously in tissue at an implant depth of between about five millimeters and about twenty millimeters, desirably in a subcutaneous pocket 94 remote from the electrode 13 and using a minimally invasive surgical procedure. As shown in FIGS. 8A and 8B, the implantation site can comprise a more medial tissue region in the lower abdomen (see also FIG. 7B). There, the pulse generator 62 can reside for extended use without causing pain and/or discomfort and/or without effecting body image. Alternatively, the implantation site can comprise a tissue region on the posterior hip, for example.

The implant system 60 includes an external patient controller-charger 64 (see FIGS. 7A and 7B). The controller 64 is sized and configured to be held by the user to transcutaneously activate and deactivate or modify the output of the pulse generator. The controller 64 may, e.g., be a simple magnet that, when placed near the site where the pulse generator 62 is implanted, toggles a magnetic switch within the implantable pulse generator 62 between an on condition and an off condition, or advances through a sequence of alternative stimulus modes pre-programmed by the clinician into implantable pulse generator 62.

Alternatively, the controller 64 may comprise more sophisticated circuitry that would allow the individual to make these selections through RF (Radio Frequency) wireless telemetry communications 66 that passes through the skin and tissue and can operate as far as an arm's length distance away from the implanted pulse generator, e.g., the controller 64 is capable of communicating with the pulse generator 62 approximately three to six feet away from the implanted pulse generator (and the pulse generator is able to communicate with the controller). The wireless telemetry circuitry provides reliable, bidirectional communications with a patient controller-charger and a clinical programmer, for example via an RF link in the 402 MHz to 405 MHz Medical Implant Communications Service (MICS) band per FCC 47 CFR Part 95, or other VHF/UHF low power, unlicensed bands.

The patient controller-charger 64 may also be belt or clothing worn and used to charge the rechargeable batteries of the pulse generator 62 as needed. Charging is achieved via an inductive RF link using a charge coil (not shown) on or near the skin in close proximity to the IPG. The patient controller-charger 64 may also be configured to provide the user with information on pulse generator battery status and stimulus levels.

When a rechargeable battery is used in the implantable pulse generator 62, the battery may have a capacity as small as about 30 mA-hr and up to about 120 ma-hr or more, and recharging of the rechargeable battery is required less than weekly. When the rechargeable battery has only a safety margin charge remaining, it can be recharged in a time period of not more than six hours.

When a primary (non-rechargeable) battery is used, the battery may have a capacity as small as about 0.5 A-hr and up to about 1.0 A-hr or more.

According to its programmed rules, when switched on, the implantable pulse generator 62 generates prescribed stimulation waveforms through the lead 12 and to the electrode 13. These waveforms bilaterally stimulate the left and right branches of the dorsal genital nerves in a manner that achieves the desired physiologic response.

It has been discovered that bilateral stimulation of the dorsal genital nerves achieved by placement of one or more electrodes 13 at a unique location in the body (which will be described in greater detail later), achieves the treatment of a variety of pelvic region disorders, such as consistently and effectively inhibiting unwanted bladder contractions. Using the controller 64, the individual may turn on or turn off the stimulation waveforms at will or adjust the strength of the waveforms.

Feasibility study results have shown significant benefits in all endpoints. For example, 21 females were enrolled in a feasibility study with a one week trial usage of a representative trial system 10. Improvements identified in the study include: leaks per day reduced in 79% of reporting subjects; heavy leakage reduced in 92% of reporting subjects; pads changed per day reduced in 83% of reporting subjects; pad weight reduced in 88% of reporting subjects; frequency reduced in 72% of reporting subjects; and severe urgency reduced in 88% of reporting subjects. The study also confirmed the lead 12 and electrode 13 can implanted with a minimally invasive pre-pubic approach, and is well tolerated by the subjects. The physicians require minimal training to perform the implant procedure, which may be performed without fluoroscopy.

A clinical programmer 68 may be used by a physician or clinician to program the pulse generator 62 with a range of preset stimulus parameters (see FIG. 22). The user will then turn the implant system On/Off using the wireless patient controller-charger 64. The controller-charger is then programmed by the pulse generator, i.e., the range of or a subset of the preset stimulus parameters previously downloaded by the clinical programmer 68 is uploaded to the controller-charger 64. This range of preset stimulus parameters allows the user to make adjustments to the stimulus strength within the preset range. Stimulation will be delivered at a level that is initially set at, below, or above the sensory threshold of the user, but is not uncomfortable. The user may get accustomed to the stimulation level, and may adjust the stimulation up or down within the preset range.

The clinical programmer 68 may be of the type described in co-pending U.S. patent application Ser. No. 11/541,890, filed Oct. 2, 2006, and entitled “Systems and Methods for Clinician Control of Stimulation Systems,” which is incorporated herein by reference. The clinical programmer 68 can be placed into transcutaneous communication with the implanted pulse generator 62, e.g., through wireless telemetry 66 that provides reliable, bidirectional communications with the programmer 68 (see FIG. 7A). The clinical programmer 68 may incorporate a custom program operating on a handheld computer or other personal digital appliance (PDA). The clinical programmer 68 or PDA includes an on-board microprocessor powered by a rechargeable, on-board battery (not shown). The microprocessor carries embedded code which may include pre-programmed rules or algorithms that allow a clinician to remotely (i.e., wirelessly) download program stimulus parameters and stimulus sequences parameters into the pulse generator. The microprocessor of the clinical programmer 68 is also desirably able to interrogate the pulse generator and upload operational data from the implanted pulse generator.

### III. The Anatomic Landmarks

As already described, components of both the trial system 10 and/or the implantable system 60 are well suited for placement and/or implantation in adipose tissue in a particular location in an individual's lower abdomen, where it has been discovered that effective bilateral stimulation of both the left and right branches of the dorsal genital nerves can be achieved with one or more electrodes. The main anatomic landmark guiding the unique placement of these components is the pubic symphysis, which offers a simple procedure for implanting a lead and electrode.

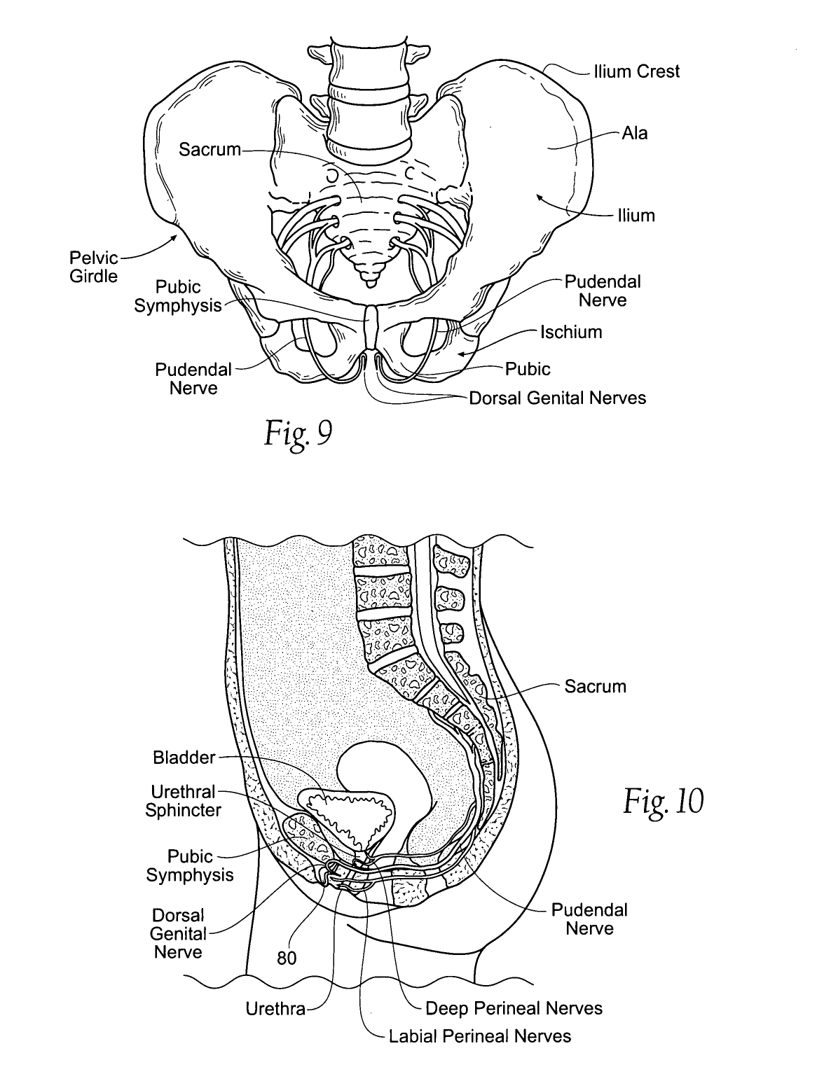

As FIG. 9 shows, the hip bones are two large, irregularly shaped bones, each of which develops from the fusion of three bones, the ilium, ischium, and pubis. The ilium is the superior, fan-shaped part of the hip bone. The ala of the ilium represents the spread of the fan. The iliac crest represents the rim of the fan. It has a curve that follows the contour of the ala between the anterior and posterior superior iliac spines.

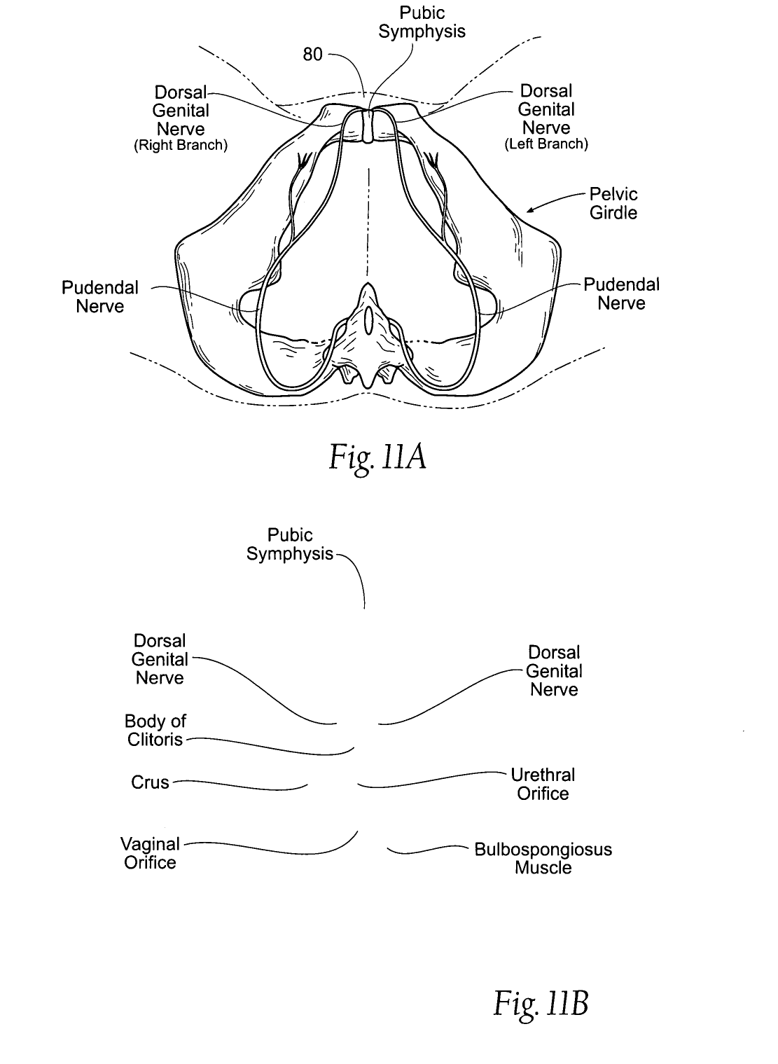

As FIGS. 9 and 10 show, the sacrum is formed by the fusion of five originally separate sacral vertebrae. The hip bones are joined at the pubic symphysis anteriorly and to the sacrum posteriorly to form the pelvic girdle (see FIG. 9). The pelvic girdle is attached to the lower limbs. Located within the pelvic girdle are the abdominal viscera (e.g., the ileum and sigmoid colon) and the pelvic viscera (e.g., the urinary bladder and female reproductive organs such as the uterus and ovaries).

Within this bony frame (see FIGS. 9 and 10), the pudendal nerve is derived at the sacral plexus from the anterior divisions of the ventral rami of S2 through S4. The pudendal nerve extends bilaterally, in separate branches on left and right sides of the pelvic girdle. Each branch accompanies the interior pudendal artery and leaves the pelvis through the left and right greater sciatic foramens between the piriformis and coccygeus muscles. The branches hook around the ischial spine and sacrospinous ligament and enter the skin and muscles of the perineum through the left and right lesser sciatic foramen.

As shown in the inferior pelvic view of FIG. 11A, the bilateral left and right branches of the pudendal nerve extend anteriorly through the perineum, each branching into the dorsal genital nerve of the penis or clitoris. The dorsal genital nerve has been found to pierce the perineal membrane generally about 2 to 4 cm lateral to the external urethral orifice, and travel along the bulbospongiousus muscle posterior to the crura and then hook over from medial to lateral to travel on the antero-lateral surface of the body of the clitoris (in females), see FIG. 11B, or extend into the body of the penis (in males). The genital nerves are the chief sensory nerve of the external genitalia. The Figures are largely based upon the anatomy of a female, but the parts of the male perineum are homologues of the female.

As FIGS. 10 and 11A show, in the male and female, adipose tissue 80 overlays the pubic symphysis. The bilateral branches of the genital nerves innervate this tissue region. In the female, this tissue region is known as the mons pubis. In the male, the penis and scrotum extend from this region.

Stimulation of the dorsal genital nerves provides direct and selective activation to the sensory fibers that lead to inhibition of the bladder and does not activate other nerve fibers that are present in the pudendal nerve and sacral spinal nerve roots. Access to the dorsal genital nerve near the pubic symphysis can be accomplished in a minimally invasive and less complicated manner and uses anatomical landmarks and structures of which pelvic health care specialists are expert, as they commonly operate in the pelvic region.

Direct stimulation of the dorsal genital nerve (a purely sensory nerve) should eliminate the variability associated with placement and stimulation of mixed (motor and sensory) nerve bundles (i.e., spillover stimulation to unwanted nerves is eliminated).

These simpler anterior surgical implantation procedures of the present invention avoid risk of injury to the spine associated with sacral nerve stimulation. The procedures may be performed without fluoroscopy or urodynamics, as the patient's report of sensation and the anatomical landmarks are used to guide placement. Implantation in the described region is in an area in which urologists commonly operate. Further, the approach is less invasive than a deep pelvic approach required to place the BION.

The placement of the lead/electrode will stimulate bilateral branches of the dorsal genital nerves, since the electrode will be placed at or near the distal end of the right and left branches. This electrode placement differs from the sacral and pudendal nerve stimulation devices that only unilaterally stimulate the left or right branch of the targeted mixed nerve, but not both.

### IV. Implantation Methodology

Representative anterior surgical techniques will now be described to place a lead 12 and electrode 13 in a desired location in adipose tissue 80 at or near the pubic symphysis. It is this desired placement that makes possible the bilateral stimulation of both left and right branches of the dorsal genital nerves with a single lead 12 to treat pelvic region disorders.

These representative surgical implantation methods for implanting the lead 12 and electrode 13, and pulse generator 62, of the present invention allows for more rapid and simple placement of these components for trial and/or longer care treatment of pelvic region disorders, whereby the electrode(s) 13 is placed so as to achieve bilateral stimulation of both left and right branches of the dorsal genital nerves. Implanting the lead 12 and electrode 13 near the dorsal genital nerves can be easily achieved without fluoroscopy, and because of this readily accessible location, implantation times are reduced from current procedures for existing neurostimulation systems stimulating nerves of the pelvic region.

Before implantation, and at the physician's discretion, an oral broad spectrum antibiotic may be given and continued for five days. With the patient in a supine or lithotomy position, the lower abdomen from the pubic symphysis to umbilicus and from the anterior iliac spines bilaterally are prepped for surgery, e.g., with Betadine (or Hibiclens Solutions for cases of Betadine allergy).

A. The Trial Stage

The trial stage installs the lead 12 and electrode 13, and connects the lead 12 to the temporary external pulse generator 14. If the use of the external pulse generator 14 achieves the desired results, the implantable pulse generator 62 is implanted in a second phase.

Local anesthesia—e.g., 1% Lidocaine (2-5 ccs) or equivalent—may be injected prior to making the anticipated insertion site 50. The site for a needle insertion is desirably located midline or near-midline, over the pubic symphysis.

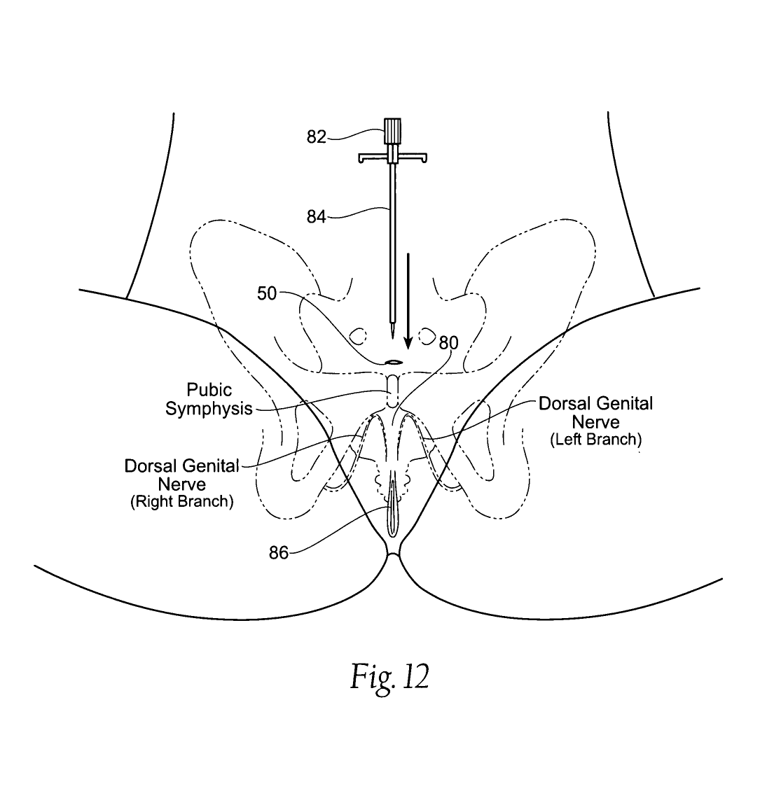

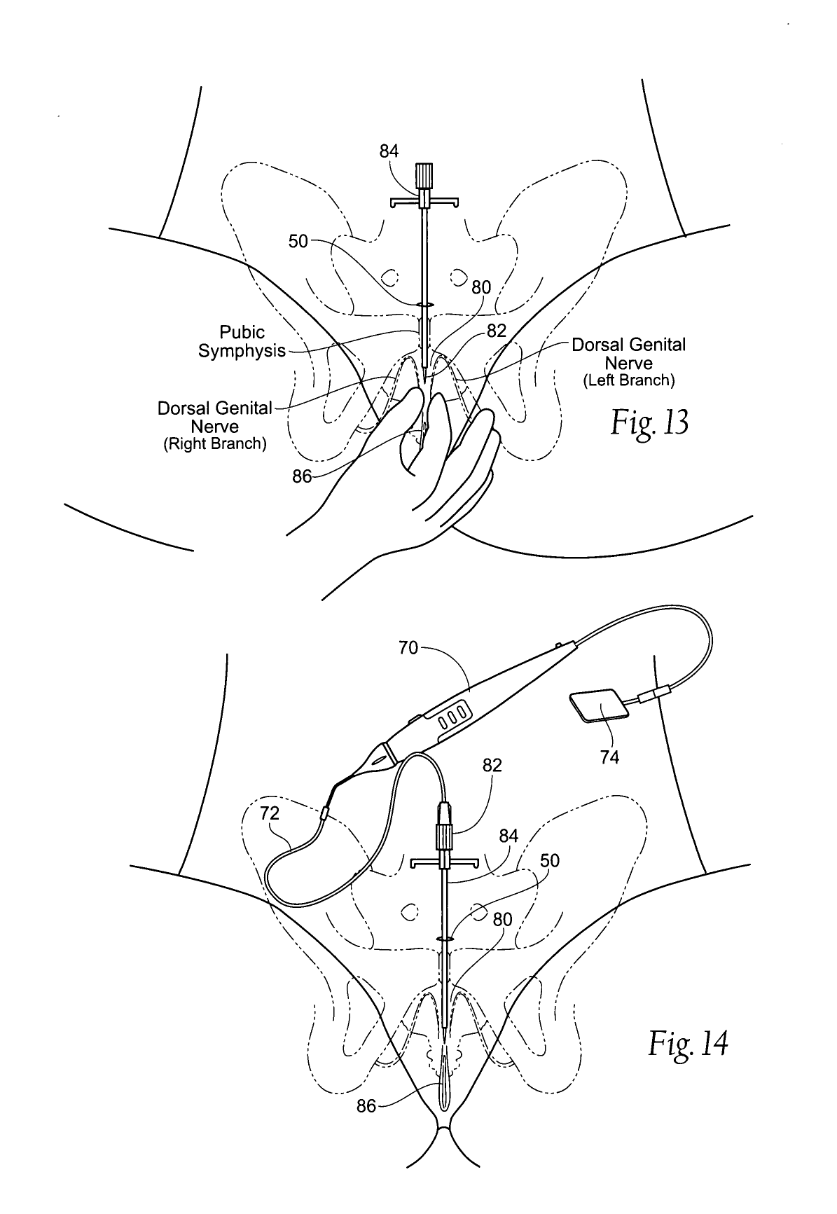

Once local anesthesia is established, and as shown in FIGS. 12 and 13, a needle 82 and sleeve 84 (the sleeve 84 being pre-loaded over the needle 82) may be used and advanced percutaneously into the anesthetized site 50 to a depth of about 0.5 centimeters to about 1.5 centimeters, and more desirably to about one centimeter. The needle 82 and sleeve 84 may then be turned caudad at an appropriate angle and passed above the pubic symphysis aiming toward the clitoris (or the base of the penis in males). The needle 82 and sleeve 84 are advanced about five centimeters to about ten centimeters necessary to reach the target site between the pubic symphysis and the clitoris 86. It is to be appreciated that these approximate insertion depths may vary depending on the particular anatomy of the patient. The physician may use one hand to guide the needle 82 and the other hand to hold the clitoris 86 to stabilize the surrounding tissue. As FIG. 14 shows, once the needle 82 is positioned, it is coupled to the test stimulator 70 through the cable 72 to apply stimulation waveforms through the needle tip concurrent with positioning of the needle 82. A patch electrode 74 placed on the skin near the hip of the individual is also coupled to the test stimulator 70 to serve as a return path for the stimulation waveforms.

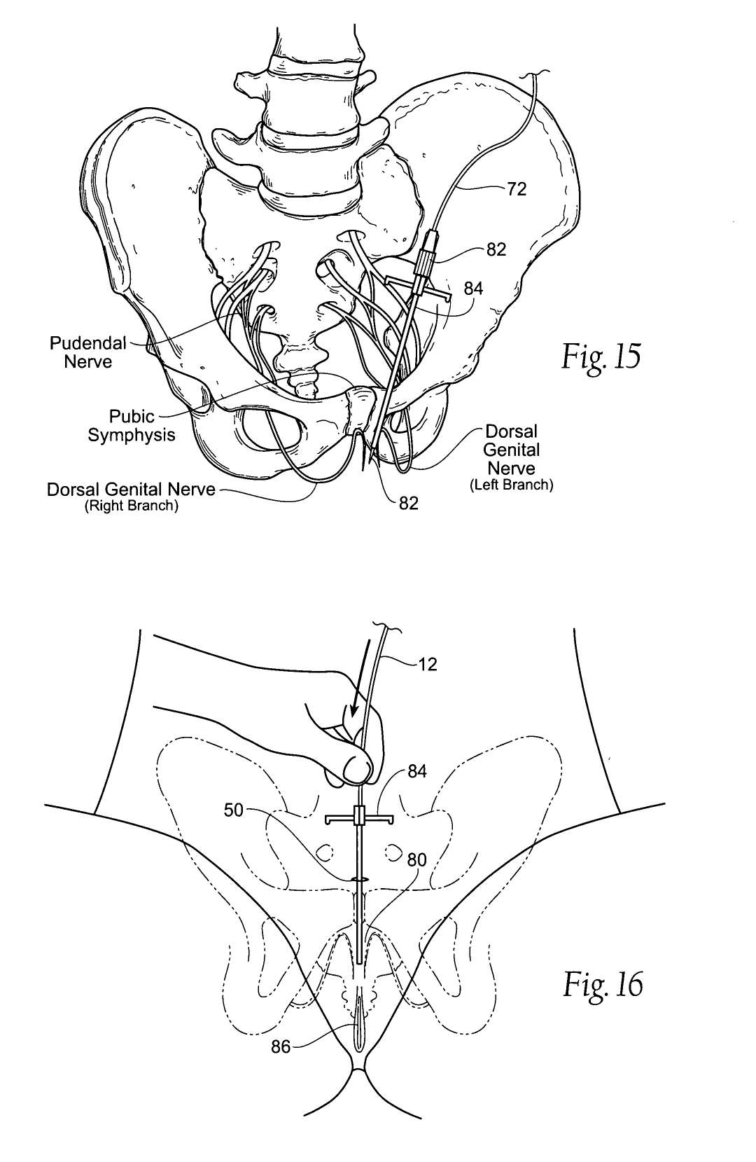

The test stimulator 70 may be used by the physician in the sterile field. The physician slowly turns up the stimulus on the test stimulator 70 and asks the patient a number of questions to elicit feedback on what they feel and where they feel the stimulation sensations. The desired sensation can be described as a thumping, tapping, or buzzing sensation near the clitoris 86. The physician may continue to ask the patient questions and to penetrate and withdraw the needle 82 and sleeve 84 (or lead 12) as necessary in a minimally invasive way, until a subcutaneous location where bilateral stimulation of both left and right branches of the genital nerves results (see FIG. 15).

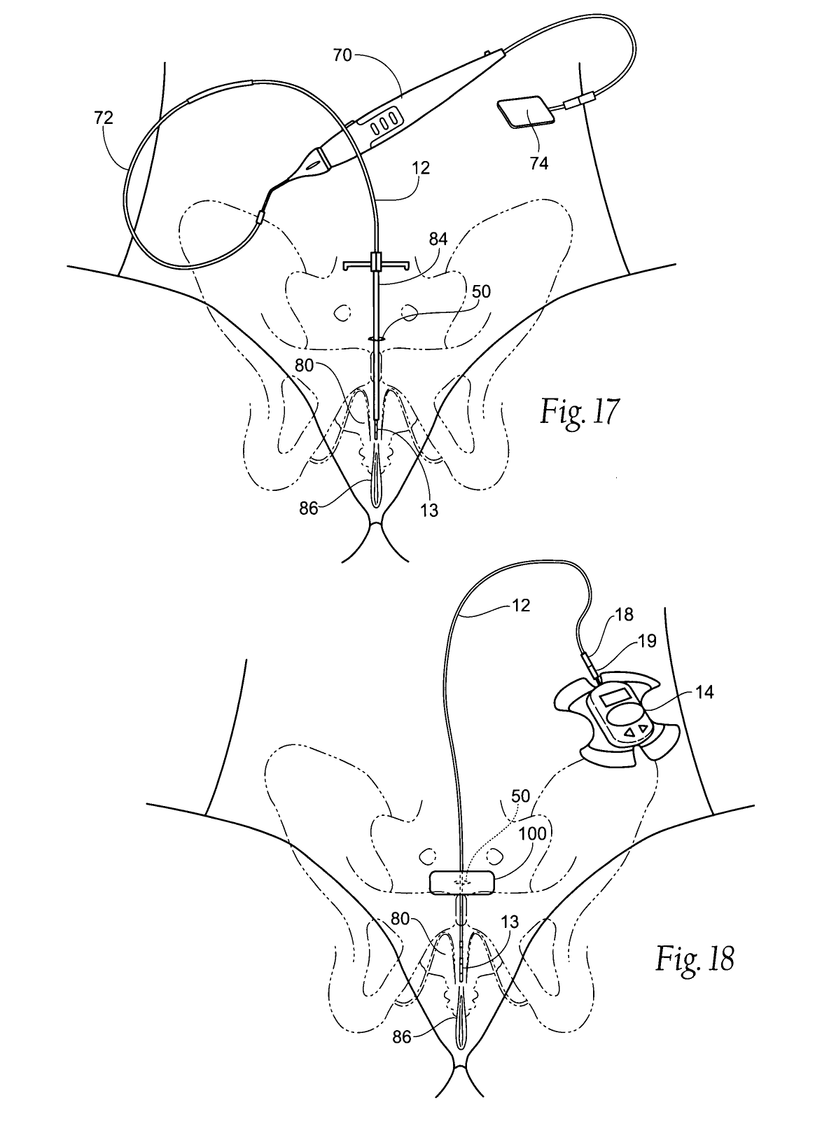

After the test stimulator 70 is disconnected from the needle 82, and the needle is removed, the lead 12, electrode-first, is passed through the sleeve 84 (see FIG. 16). A guide wire may be preloaded into a lumen in the lead 12 to provide temporary stiffening during insertion (not shown). The lead 12 is now coupled to the test stimulator 70 to again apply stimulation waveforms through the electrode 13 concurrent with positioning of the electrode (see FIG. 17). Again, the physician slowly adjusts the stimulation via the test stimulator 70 and asks for the patient feedback of sensation. Based on the patient feedback, the physician repositions the lead if necessary.

Once the optimal location is found, the physician removes the cable 72 from the lead 12, and applies pressure on the skin over top where the electrode 13 is positioned and withdraws the sleeve 84. The guide wire may be withdrawn from the lead 12. The applied pressure helps to maintain the lead 12 in place while the sleeve 84 is being removed. The patch electrode 74 may be removed, and the test stimulator 70 and the patch electrode may be discarded.

Optionally, the test stimulator 70 may again be coupled to the lead 12 via the cable 72 to apply stimulation pulses through the electrode 13, to confirm that the electrode 13 resides in the location previously found.

After the electrode 13 and a portion of the lead 12 is implanted as described above, and with a portion of the lead 12 extending out of the skin, plug 18 may be connected to the external pulse generator 14 (as FIG. 18 shows), and the external pulse generator 14 may be applied to the skin. The lead 12 may then be secured externally to the skin with a piece of TEGADERM™ dressing or sterile tape 100, for example, which may also cover the lead insertion site 50. Additional pieces may be used as necessary. The sterile tape 100 covering the insertion site 50 and the re-growth of tissue maintains this sterile barrier.

B. The Second Phase

As previously described, the second phase may be warranted if there is an improvement in the treated disorder. Alternatively, the trial system 10 may be used for longer-term care treatment if the implanting the implantable system 60 is not desirable.

The patient is again prepared for a surgical procedure. Known preoperative antibiotics and skin prep may be performed. Under local anesthesia, the lead 12 and electrode 13 is located as previously described for the trial stage of the two phase procedure, and as shown in FIGS. 12 through 18.

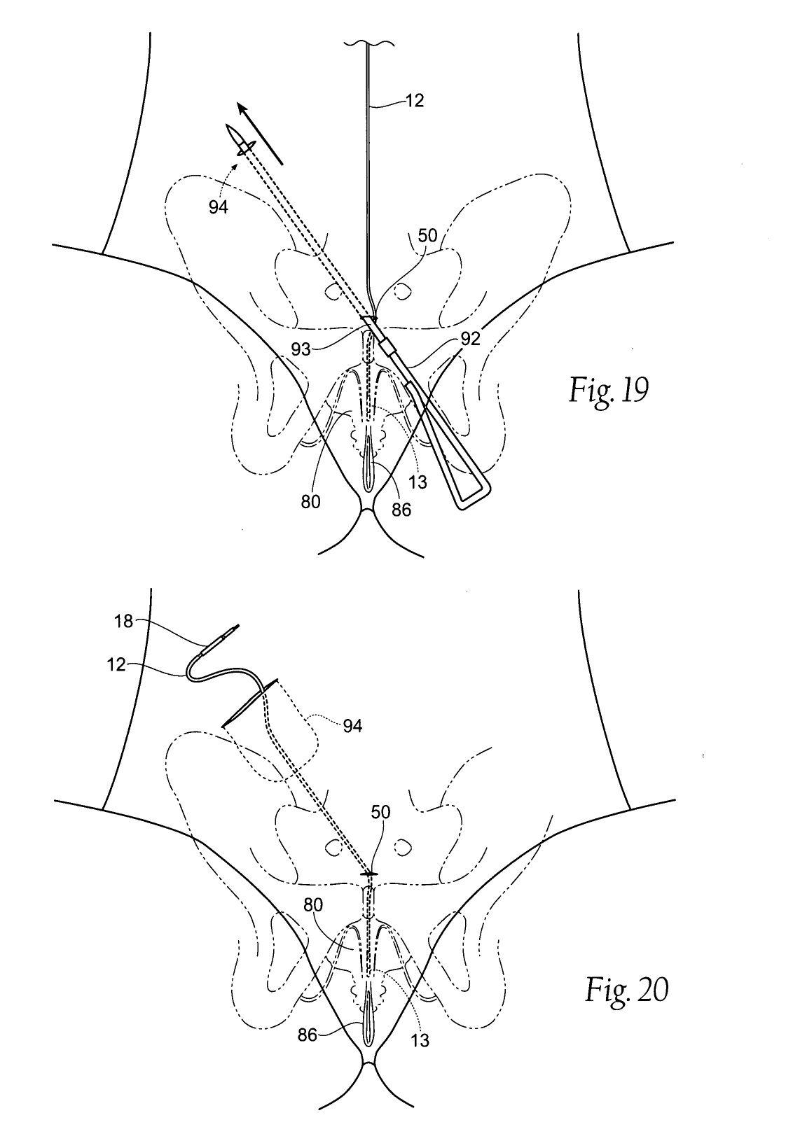

For the tunneling procedure, the patient may undergo monitored anesthesia care (MAC), which is a planned procedure during which the patient generally undergoes local anesthesia together with sedation and analgesia. During MAC, the patient is sedated and amnestic but always remains responsive when stimulated to do so. Having implanted the lead/electrode, and under MAC and/or local anesthesia, a subcutaneous tunnel is formed for connecting the lead 12 to the pulse generator 62. A tunneling tool 92 having a sleeve 93 is manipulated by the physician to route (tunnel) the lead 12 subcutaneously to a pocket site 94 where the pulse generator 62 is to be implanted (see FIG. 19).

After placement of the lead 12 as FIG. 20 shows, the subcutaneous pocket 94 is formed to accept the pulse generator 62 using blunt dissection techniques of the subcutaneous tissues.

With the pocket 94 formed, and the lead 12 and plug 18 delivered into the procedural field, the lead can now be connected to the pulse generator 62.

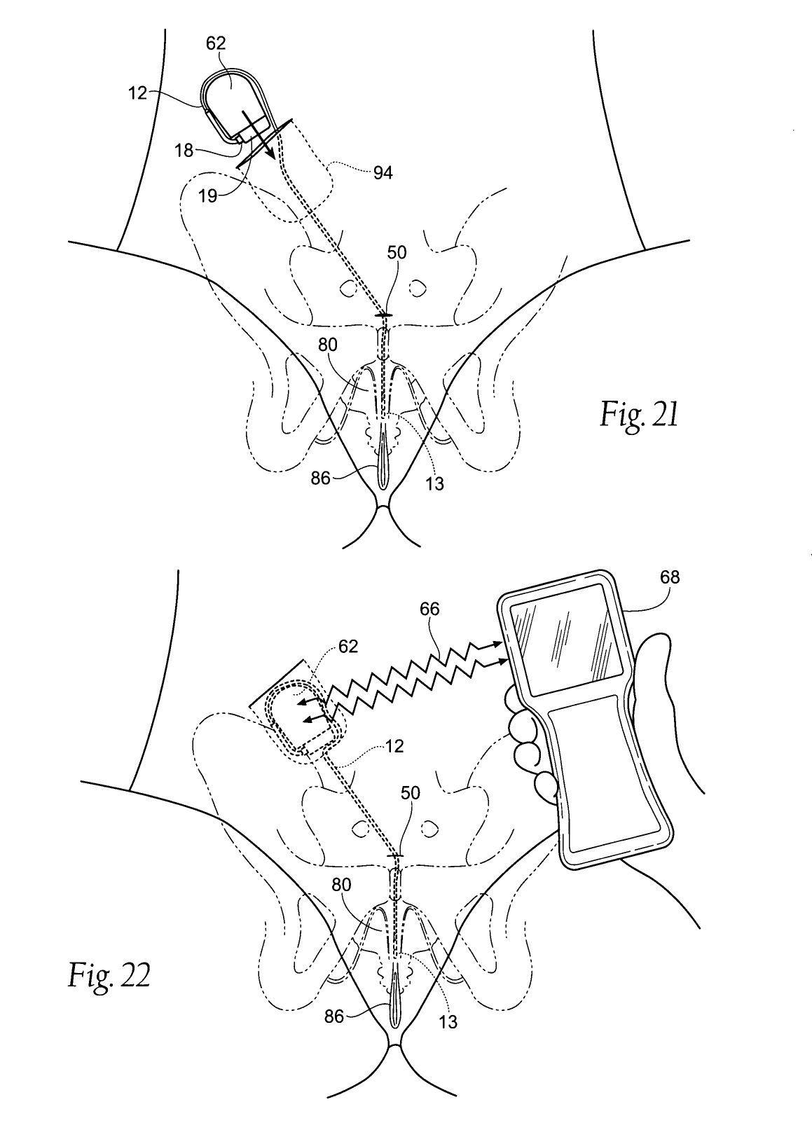

Once the lead 12 has been connected to the pulse generator 62, the lead 12 and pulse generator can be placed into the pocket 94 (see FIG. 21).

As can be seen in FIG. 22, the clinical programmer 68 is used to turn on the pulse generator 62 and to test the stimulus response. The clinical programmer would use wireless telemetry and may be located either inside or outside of the surgical field, e.g., up to about three to six feet away from the implanted pulse generator 62.

At the physician's discretion, some or all of the wound sites may be irrigated with irrigation solutions (e.g., ½ strength betadine or Hibiclens solution), and closed using DERMABOND® glue, STERI-STRIP® material, or stitches of 4-0 VICRYL®, for example. Dressing is desirably applied for about twenty-four hours. The incisions are desirably kept dry for forty-eight hours.

### V. Kits

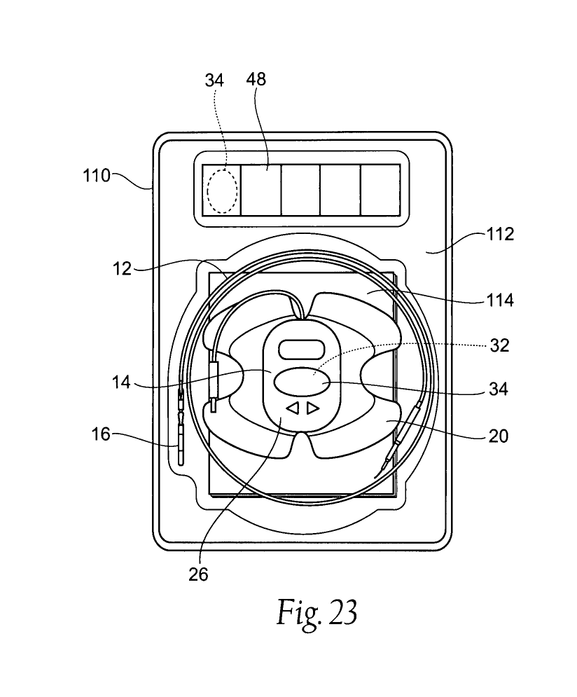

Various components, assemblies, tools, and systems as just described can be consolidated for use in functional kits. An exemplary kit 110 is shown in FIG. 23 and can take various forms. In the illustrated embodiment, kit 110 comprises a sterile, wrapped assembly. Kit 110 includes an interior tray 112 made, e.g., from die cut cardboard, plastic sheet, or thermo-formed plastic material, which hold the contents. Kit 110 also may include instructions 114 for using the contents of the kit to carry out a desired procedure and/or to screen and/or treat diverse therapeutic and functional restorations indications, as described herein.

As shown in FIG. 23, the exemplary kit 110 includes components adapted to screen and/or treat pelvic region disorders, while other kits may include tools used in the surgical procedures described. The kit 110 is shown including a lead 12 including a proximal portion, and an electrode 16 at or near a distal portion. The kit is also shown to include an external pulse generator 14, the external pulse generator comprising a carrier 20 adapted to be worn by a user, and a removable and replaceable electronics pod 26 adapted to be coupled to the carrier 20, the electronics pod 26 containing circuitry and adapted to generate electrical stimulation current patterns to be delivered through the percutaneous lead 12 and to the electrode 16 to stimulate tissue. The electronics pod 26 includes a power input bay 32 adapted to receive a self-contained, limited life, disposable, smart power source 34 that can be released and replaced for a prescription period, the power source 34 including circuitry to provide power source information to the electronics pod 26 and/or receive power source information from the electronics pod 26.

The kit 110 also includes one or more power sources 34, each power source comprising a dose of power for the circuitry for administration according to a prescribed power source replacement regime. The supply of power sources may be provided in a power source organizer 48 that includes a compartment for each prescribed repeated basis, the compartment adapted to hold one or more power sources.

Instructions are provided for use prescribing the release and replacement of the power source according to the prescribed power source replacement regime, the prescribed power source replacement regime comprising the replacement of the power source on a prescribed repeated basis similar to administering a pill under a prescribed pill-based medication regime.

The kit 110 also includes instructions for implanting the electrode in tissue in a region at or near a pubic symphysis, coupling the lead to the external pulse generator, and stimulating the left and/or right branches of the dorsal genital nerves by conveying electrical stimulation waveforms from the external pulse generator to the electrode to screen and/or treat pelvic region disorders.

The instructions 114 can, of course vary. The instructions 114 shall be physically present in the kits, but can also be supplied separately. The instructions 114 can be embodied in separate instruction manuals, or in video or audio tapes, CD's, and DVD's. The instructions 114 for use can also be available through an internet web page.

### VI. Representative Indications

Due to their technical features, the trial system 10 and implantable system 60 can be used to screen and/or treat diverse therapeutic and functional restorations indications.

For example, in the field of pelvic region disorders (i.e., urology), possible indications for use of the systems 10 and 60 include the treatment of urinary incontinence (including at least stress and urge incontinence), overactive bladder, neurogenic bladder, micturition disorders (including at least urinary retention), defecation disorders (including at least fecal incontinence and constipation), sexual disorders, pelvic floor muscle disorders, prostate disorders, and pelvic pain disorders (including at least interstitial cystitis and painful bladder syndrome).

The systems 10 and 60 can be used for deep brain stimulation in the treatment of (i) Parkinson's disease; (ii) multiple sclerosis; (iii) essential tremor; (iv) depression; (v) eating disorders; (vi) epilepsy; and/or (vii) minimally conscious state.

The systems 10 and 60 can be used for pain management by interfering with or blocking pain signals from reaching the brain, in the treatment of, e.g., (i) peripheral neuropathy; and/or (ii) cancer.

The systems 10 and 60 can be used for vagal nerve stimulation for control of epilepsy, depression, or other mood/psychiatric disorders.

The systems 10 and 60 can be used for the treatment of obstructive sleep apnea.

The systems 10 and 60 can be used to aid in fertility.

The systems 10 and 60 can be used for gastric stimulation to prevent reflux or to reduce appetite or food consumption.

The systems 10 and 60 can be used to compensate for various cardiac dysfunctions, such as rhythm disorders.

The systems 10 and 60 can be used in functional restorations indications such as the restoration of motor control, to restore (i) impaired gait after stroke or spinal cord injury (SCI); (ii) impaired hand and arm function after stroke or SCI; (iii) respiratory disorders; (iv) swallowing disorders; (v) sleep apnea; and/or (vi) neurotherapeutics, allowing individuals with neurological deficits, such as stroke survivors or those with multiple sclerosis, to recover functionally.

The systems 10 and 60 can be used for veterinary uses. The ability to control/activate sexual actions such as erection and/or ejaculation actions may be used in animal reproduction technologies, such as artificial insemination. Artificial insemination is commonly used for selective reproduction of bovines, swine, horses, dogs, and cats, as non-limiting examples.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A method for screening and/or treating a pelvic region disorder comprising
providing an external pulse generator,
providing a percutaneous lead including a connector at or near a proximal portion, and an electrode at or near a distal portion, the electrode being electrically coupled to the connector,
inserting the distal portion of the percutaneous lead into tissue near-midline over the pubic symphysis, positioning the electrode at a target site between the pubic symphysis and the clitoris of a female or the base of the penis of a male,
coupling the percutaneous lead to the external pulse generator, and
operating the external pulse generator to convey stimulation waveforms to the electrode to screen and/or treat the pelvic region disorder.

2. A method according to claim 1
wherein the target site between the pubic symphysis and the clitoris of a female or the base of the penis of a male comprises an adipose tissue region innervated with one or more nerves.

3. A method according to claim 1
wherein the stimulation waveforms conveyed to the electrode affect stimulation of the left or right branches of the dorsal genital nerves.

4. A method according to claim 1
wherein the stimulation waveforms conveyed to the electrode affect bilateral stimulation of the left and right branches of the dorsal genital nerves.

5. A method according to claim 1
wherein the pelvic region disorder is selected from the group consisting of urinary incontinence, overactive bladder, neurogenic bladder, micturition disorders, defecation disorders, sexual disorders, prostate disorders, pelvic floor muscle disorders, and pelvic pain.

6. A method according to claim 1
wherein the step of providing an external pulse generator comprises providing a removable and replaceable carrier adapted to be worn by a user, and
providing a removable and replaceable electronics pod to electrically couple to the carrier, the electronics pod comprising circuitry adapted to generate electrical stimulation current patterns to be delivered through the percutaneous lead and to the electrode, the electronics pod also comprising a power input bay adapted to receive a self-contained, limited life, disposable, smart power source that can be released and replaced for a prescription period, the power source including circuitry to provide power source information to the electronics pod and/or receive power source information from the electronics pod.

7. A method for screening and/or treating a pelvic region disorder comprising
providing an external pulse generator,
providing a percutaneous lead including a proximal portion, and an electrode at or near a distal portion,
providing a test stimulator, the test stimulator comprising a hand-held, single use, sterile, and disposable device including test stimulation generating circuitry and a non-rechargeable and non-replaceable battery adapted to keep the test stimulator operational for a predetermined time,
implanting the distal portion of the percutaneous lead near-midline over the pubic symphysis to position the electrode at a target site between the pubic symphysis and the clitoris of a female or the base of the penis of a male,
coupling the test stimulator to the proximal portion of the percutaneous lead,
operating the test stimulator to deliver test stimulation to the electrode to confirm the electrode is positioned at the target site,
adjusting the position of the electrode if necessary to confirm the electrode is positioned at the target site,
after confirming that the electrode is positioned at the target site, uncoupling the test stimulator from the percutaneous lead,
coupling the percutaneous lead to the external pulse generator,
operating the external pulse generator to convey stimulation waveforms through the lead to the electrode to screen and/or treat the pelvic region disorder, and discarding the test stimulator.

8. A method according to claim 7
wherein the stimulation waveforms conveyed to the electrode affect stimulation of the left and/or right branches of the dorsal genital nerves.

9. A method according to claim 7
wherein the pelvic region disorder is selected from the group consisting of urinary incontinence, overactive bladder, neurogenic bladder, micturition disorders, defecation disorders, sexual disorders, prostate disorders, pelvic floor muscle disorders, and pelvic pain.

10. A method according to claim 7
wherein the implanting step is performed without fluoroscopy.

11. A method for providing neurostimulation to tissue comprising:
providing a percutaneous lead including a proximal portion and a distal portion, the distal portion including one or more electrodes,
providing an external pulse generator adapted to electrically couple to the percutaneous lead and one or more electrodes, the external pulse generator comprising a carrier adapted to be worn by a user,
providing a removable and replaceable electronics pod to electrically coupled to the carrier, the electronics pod comprising circuitry adapted to generate electrical stimulation current patterns to be delivered through the percutaneous lead and to the at least one electrode to stimulate tissue, the electronics pod also comprising a power input bay adapted to receive a self-contained, limited life, disposable, smart power source that can be released and replaced for a prescription period, the power source including circuitry to provide power source information to the electronics pod and/or receive power source information from the electronics pod,
providing instructions for use prescribing the release and replacement of the power source according to a prescribed power source replacement regime, the prescribed power source replacement regime comprising the replacement of the power source on a prescribed repeated basis similar to administering a pill under a prescribed pill-based medication regime,
providing a supply of power sources, each power source comprising a dose of power for the circuitry for administration according to the prescribed power source replacement regime,
implanting the distal portion of the percutaneous lead on, in, or near a targeted tissue region, coupling the percutaneous lead to the external pulse generator,
inserting a power source into the power input bay, the power source circuitry providing power source information to the electronics pod and/or receiving power source information from the electronics pod, and
operating the external pulse generator to convey stimulation waveforms to the targeted tissue region to provide neurostimulation.

12. A method according to claim 11
wherein the targeted tissue region comprises the left and/or right branches of the dorsal genital nerves, the pudendal nerve and/or its branches, the perineal nerves, and/or its branches, the urethral nerves, and/or its branches, and/or the sacral nerves.

13. A method according to claim 11
wherein the power source information comprises the power capacity of the power source, or the total power usage provided to date by the power source, or the total power capacity remaining in the power source, or a unique identification to identify the power source, or any combination.

14. A method according to claim 11
wherein the supply of power sources comprises the supply of power sources and a power source organizer that includes a compartment for each prescribed repeated basis, the compartment adapted to hold one or more power sources.

15. A method according to claim 11
wherein the external pulse generator provides stimulus waveforms to screen and/or treat indications, the indications selected from the group consisting of pelvic region disorders, deep brain stimulation, pain management, vagal nerve stimulation, obstructive sleep apnea, fertility, gastric stimulation, cardiac dysfunctions, functional restorations indications, and veterinary indications.

16. A neurostimulation system for screening and/or treatment of pelvic region disorders comprising:
a percutaneous lead including a proximal portion and a distal portion, the proximal portion including a connector, the distal portion able to reside in adipose tissue and including an electrode, the electrode being electrically coupled to the connector,
an external pulse generator adapted to electrically couple to the percutaneous lead, the external pulse generator comprising a removable and replaceable carrier adapted to be worn by a user, the carrier including a tissue facing surface having pressure sensitive adhesive to removably secure the carrier to the user's skin, a removable and replaceable electronics pod electrically coupled to the carrier, the electronics pod containing circuitry and adapted to generate electrical stimulation current patterns to be delivered through the percutaneous lead and to the electrode to stimulate tissue, and
a power input bay adapted to receive a self-contained, limited life, disposable, smart power source that can be released and replaced for a prescription period, the power source including circuitry to provide power source information to the electronics pod and/or receive power source information from the electronics pod.

17. A system according to claim 16
wherein the power source circuitry comprises a non-volatile memory to store the power source information.

18. A system according to claim 16
wherein the supply of power sources comprises a power source organizer that includes a compartment for each prescription period, the compartment adapted to hold one or more power sources.

19. A system according to claim 16
wherein the tissue facing surface includes a return electrode to couple to the user's skin.

20. A system according to claim 16
wherein the pelvic region disorder is selected from the group consisting of urinary incontinence, overactive bladder, neurogenic bladder, micturition disorders, defecation disorders, sexual disorders, prostate disorders, pelvic floor muscle disorders, and pelvic pain.

21. A kit of devices to screen and/or treat a pelvic region disorder comprising:
a percutaneous lead including a proximal portion, and an electrode at or near a distal portion,
an external pulse generator comprising a carrier adapted to be worn by a user and a removable and replaceable electronics pod adapted to be coupled to the carrier, the electronics pod containing circuitry and adapted to generate electrical stimulation current patterns to be delivered through the percutaneous lead and to the electrode to stimulate tissue, the electronics pod including a power input bay adapted to receive a self-contained, limited life, disposable, smart power source that can be released and replaced for a prescription period, the power source including circuitry to provide power source information to the electronics pod and/or receive power source information from the electronics pod,
one or more power sources, each power source comprising a dose of power for the circuitry for administration according to a prescribed power source replacement regime,
instructions for use prescribing the release and replacement of the power source according to the prescribed power source replacement regime, the prescribed power source replacement regime comprising the replacement of the power source on a prescribed repeated basis similar to administering a pill under a prescribed pill-based medication regime, and
instructions for implanting the electrode in tissue in a region at or near a pubic symphysis, coupling the lead to the external pulse generator, and stimulating the left and/or right branches of the dorsal genital nerves by conveying electrical stimulation waveforms from the external pulse generator to the electrode to screen and/or treat the pelvic region disorder.

22. A kit according to claim 21
wherein the power source information comprises the power capacity of the power source, or the total power usage provided to date by the power source, or the total power capacity remaining in the power source, or a unique identification to identify the power source, or any combination.

23. A kit according to claim 21
further including a supply of power sources, the supply of power sources comprises a power source organizer that includes a compartment for each prescribed repeated basis, the compartment adapted to hold one or more power sources.

24. A kit according to claim 21
wherein the electrical connection region on the electronics pod electrically couples to the mating electrical connection region on the carrier to electrically couple the circuitry on the electronics pod to a return electrode positioned on the tissue facing surface of the carrier.

25. A kit according to claim 21
wherein the system includes a patch return electrode to couple to the electronics pod.
